(19) United States
(12) Patent Application Publication
(10) Pub. No.: US 2013/0166000 A1
(43) Pub. Date: Jun. 27, 2013

(54) SETSCREW CONTACT FOR IMPLANTABLE PULSE GENERATOR

(71) Applicant: Wisit Lim, Santa Clarita, CA (US)

(72) Inventor: Wisit Lim, Santa Clarita, CA (US)

(73) Assignee: PACESETTER, INC., Sylmar, CA (US)

(21) Appl. No.: 13/333,851

(22) Filed: Dec. 21, 2011

(51) Int. Cl. A61N 1/375

(52) U.S. Cl. 607/72

(57) ABSTRACT

A header of an implantable medical pulse generator may include a lead connector end receiving receptacle for transmitting electrical pulses from the can to the lead through an electrically conductive setscrew contact in electrical communication with a terminal of the lead connector end. The setscrew contact includes a setscrew hole and a lead connector hole. The lead connector hole is aligned with the lead connector end receiving receptacle, and the setscrew hole includes a setscrew threadably received therein. Inner circumferential surfaces of the setscrew hole and lead connector hole are generally tangentially to each other such that a window is created by the overlap of the inner circumferential surfaces.

### FIELD OF THE INVENTION

Aspects of the present invention relate to medical apparatus and methods. More specifically, the present invention relates to apparatus for electrically connecting an implantable medical lead to an implantable pulse generator.

### BACKGROUND OF THE INVENTION

An implantable pulse generator such as an artificial pacemaker or implantable cardioverter defibrillator (“ICD”) is a medical device that uses electrical impulses to regulate the beating of a heart. In general, the pulse generator administers electrical impulses to the appropriate heart tissue via one or more leads inserted transvenously with distal ends of the leads located within the chamber or chambers of the heart. The distal ends of the leads support electrodes for sensing, pacing and defibrillation.

After placement of the electrodes, the proximal ends of the leads may be physically and electrically connected to the pulse generator. In some instances, the pulse generator may be placed below the subcutaneous layer of the chest. The pulse generator utilizes a battery or other power source to generate the electrical impulses which are transmitted through conductors in the leads to the electrodes at the lead distal end and into the heart for regular pacing. As such, the electrical connection between the proximal ends of the leads and the pulse generator is of significant importance to provide a steady and regular pace to the heart.

Patient comfort and medical complications (e.g., pocket erosion) associated with implantable pulse generators can be, respectively, increased and decreased by reducing the size of the implantable pulse generator and increasing the smoothness of the header sidewalls. However, since implantable pulse generators are life sustaining devices the implantable pulse generators need to have robust construction, including dependable electrical connections between the lead proximal ends and the pulse generator.

There is a need in the art for a setscrew contact that provides dependable electrical connection between a lead proximal end and a pulse generator, yet allows the pulse generator to have a reduced size and increased smoothness for the header sidewalls.

### BRIEF SUMMARY OF THE INVENTION

One implementation of the present disclosure may take the form of an implantable medical pulse generator for administering electrotherapy via an implantable medical lead having a lead connector end on a proximal end of the lead, the lead connector having a terminal. The pulse generator includes a can and a header. The header is coupled to the can and includes a lead connector end receiving receptacle and a conductive setscrew contact. The conductive setscrew contact is associated with the lead connector end receiving receptacle and configured to be in electrical contact with the terminal of the first lead connector end when the lead connector end is inserted into the lead connector end receiving receptacle. The conductive setscrew contact includes a lead connector hole, a setscrew hole and a setscrew. The lead connector hole is defined at least in part by a circumferential inner surface and includes a longitudinal axis. The lead connector hole is generally aligned with the lead connector end receiving receptacle. The setscrew hole is defined at least in part by a circumferential inner surface and includes a longitudinal axis generally perpendicular to and offset away from the longitudinal axis of the lead connector hole. The circumferential inner surface of the setscrew hole is nearly tangential with the circumferential inner surface of the lead connector hole such that a window is defined in the circumferential inner surface of the setscrew hole and the circumferential inner surface of the lead connector hole by the overlapping of the volumes of the lead connector hole and setscrew hole. The setscrew includes a threaded shaft extending from a head. A portion of the head resides within the window to protrude from the setscrew hole into the lead connector hole when the setscrew is threadably received in the setscrew hole.

In one embodiment, a width of the window transverse to the longitudinal axis of the lead connector hole is less than a diameter of the lead connector hole. A width of the window transverse to the longitudinal axis of the setscrew hole is less than a diameter of the setscrew hole.

In one embodiment, the setscrew includes a conical portion which is the portion of the head residing with the window to protrude from the setscrew hole into the lead connector hole when the setscrew is threadably received in the setscrew hole. In one embodiment, the setscrew includes a cylindrical portion which is the portion of the head residing with the window to protrude from the setscrew hole into the lead connector hole when the setscrew is threadably received in the setscrew hole.

In one embodiment, the setscrew further includes a groove defined in an outer circumferential surface of the cylindrical portion of the head of the setscrew, the groove being occupied by an O-ring with an outer circumferential boundary that does not radially extend past the outer circumferential surface of the cylindrical portion except at radially spaced apart distinct protrusions of the O-ring that do radially extend past the outer circumferential surface of the cylindrical portion. The only part of the O-ring that contacts the inner circumferential surface of the setscrew hole when the setscrew is threadably received in the setscrew hole are the protrusions of the O-ring. The contact of the O-ring protrusions with the inner circumferential surface of the setscrew hole does not create a fluid tight seal about the setscrew head. However, the contact of the O-ring protrusions with the inner circumferential surface of the setscrew hole creates an arrangement that helps to resist the unintentional or self rotation of the setscrew within the setscrew hole, thereby preventing the setscrew from unintentionally backing out of the setscrew hole.

In one embodiment, the header further includes a setscrew bore generally aligned with the setscrew hole. Also, the header includes a septum configured to create a fluid tight seal with an inner circumferential surface of the setscrew bore, the septum being located in the setscrew bore outward from the setscrew head.

Another implementation of the present disclosure may take the form of an implantable medical pulse generator for administering electrotherapy via an implantable medical lead having a lead connector end on a proximal end of the lead, the lead connector having a terminal. The pulse generator includes a can and a header. The header is coupled to the can and includes a lead connector end receiving receptacle and a conductive setscrew contact. The conductive setscrew contact is associated with the lead connector end receiving receptacle and configured to be in electrical contact with the terminal of the first lead connector end when the lead connector end is inserted into the lead connector end receiving receptacle. The conductive setscrew contact includes a lead connector hole, a setscrew hole, and a setscrew. The lead connector hole is defined at least in part by a circumferential inner surface and includes a longitudinal axis. The lead connector hole is generally aligned with the lead connector end receiving receptacle. The setscrew hole is defined at least in part by a circumferential inner surface and includes a longitudinal axis generally perpendicular to and offset away from the longitudinal axis of the lead connector hole. The setscrew includes a threaded shaft extending from a head. A portion of the head protrudes from the setscrew hole into the lead connector hole when the setscrew is threadably received in the setscrew hole. The setscrew further includes a groove defined in an outer circumferential surface of a cylindrical portion of the head of the setscrew. The groove is occupied by an O-ring with an outer circumferential boundary that does not radially extend past the outer circumferential surface of the cylindrical portion except at radially spaced apart distinct protrusions of the O-ring that do radially extend past the outer circumferential surface of the cylindrical portion.

In one embodiment, the only part of the O-ring that contacts the inner circumferential surface of the setscrew hole when the setscrew is threadably received in the setscrew hole are the protrusions of the O-ring. The contact of the O-ring protrusions with the inner circumferential surface of the setscrew hole does not create a fluid tight seal about the setscrew head. However, the contact of the O-ring protrusions with the inner circumferential surface of the setscrew hole creates an arrangement that helps to resist the rotation of the setscrew within the setscrew hole.

In one embodiment, the header further includes a setscrew bore generally aligned with the setscrew hole. The setscrew bore includes a septum configured to create a fluid tight seal with an inner circumferential surface of the setscrew bore. The septum is located in the setscrew bore outward from the setscrew head.

While multiple embodiments are disclosed, still other embodiments of the present disclosure will become apparent to those skilled in the art from the following detailed description, which shows and describes illustrative embodiments of the disclosure. As will be realized, the invention is capable of modifications in various aspects, all without departing from the spirit and scope of the present disclosure. Accordingly, the drawings and detailed description are to be regarded as illustrative in nature and not restrictive.

### BRIEF DESCRIPTION OF THE DRAWINGS

FIG. 1 is an isometric view of a proximal end portion (i.e., lead connector end) of a conventional transvenous bipolar pacing lead.

FIG. 2 is an isometric view of a prior art cardiac pacemaker/defibrillator unit (i.e., pulse generator) incorporating connector junctions for communication with one or more electrodes.

FIGS. 3A and 3B are isometric views of a new and novel pulse generator wherein the connector assembly includes five receptacles.

FIG. 3C is an isometric view of the pulse generator of FIGS. 3A and 3B, except the components inside the exterior surface of the connector assembly are shown in hidden lines.

FIG. 4 is a side elevation view of the connector assembly of FIG. 3C, wherein the components inside the exterior surface of the connector assembly are shown in hidden lines.

FIG. 5 is a transverse cross section elevation view of the connector assembly as taken along section line 5-5 in FIG. 4, wherein the components inside the exterior surface of the connector assembly are shown in hidden lines.

FIG. 6 is an isometric view of a typical setscrew of the connector assembly of FIGS. 3-5.

FIG. 7 is a side elevation view of the setscrew of FIG. 6.

FIG. 8 is a top plan view of the setscrew of FIG. 6.

FIG. 9 is a transverse cross section through the head as taken along section line 9-9 in FIG. 7.

FIG. 10 is a longitudinal cross section through the setscrew as taken along section line 10-10 in FIG. 8.

FIG. 11 is another longitudinal cross section through the setscrew as taken along section line 11-11 in FIG. 8.

FIG. 12 is an isometric view of a typical conductive setscrew contact of the connector assembly with the setscrew shown aligned with a threaded setscrew receiving opening defined in the conductive setscrew contact.

FIG. 13 is an isometric view of the setscrew threadably received in the conductive setscrew contact.

FIG. 14 is an isometric cross sectional elevation as taken along section line 14-14 of FIG. 13.

### DETAILED DESCRIPTION

Implementations of the present disclosure involve an implantable medical pulse generator for administering electrotherapy via an implantable medical lead having a lead connector end on a proximal end. The pulse generator may include a can and a header coupled to the can. The header may include a first lead connector end receiving receptacle for transmitting electrical pulses from the can to the lead through one or more electrical contacts in electrical communication with one or more terminals of the lead connector.

In one embodiment, an electrical contact may be a setscrew electrical contact 47 wherein the contact includes a setscrew hole 82 and a lead connector hole 84. A window 90 is defined in the cylindrical boundary walls 96, 102 defining the respective holes 82, 84. The window 90 is the result of the overlap of the two cylindrical volumes of the holes 82, 84 defined by the cylindrical boundary walls 96, 102. A portion 66 of the setscrew head 58 increasingly protrudes into the volume of the lead connector hole 84 as the setscrew 56 is increasingly threaded into the setscrew hole 82. As a result, the setscrew causes an impingement of the lead connector end tip or pin contact 12 between the setscrew head 58 and the inner circumferential surface 102 of the lead connector hole 84, thereby mechanically securing the lead connector end 10 within the receptacle 32 of the header 22 of the pulse generator 20 and creating an electrical contact between the tip or pin contact 12 and the electrical components of the pulse generator 20.

The setscrew contact disclosed herein is advantageous in that it provides a dependable electrical connection between a lead proximal end and a pulse generator while allowing the pulse generator to have a reduced size and generally smooth surface and substantially planar side surfaces. As a result, patient comfort is increased and medical complications are reduced.

Before a detailed discussion of the contact assembly of the header is provided, a general discussion is first given regarding common features of a common lead connector end at the proximal end of an implantable medical lead followed by a general discussion of the features of an implantable medical pulse generator.

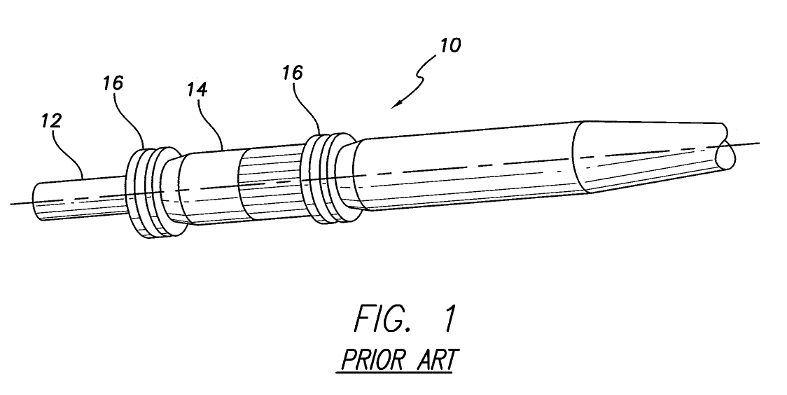

FIG. 1 shows a proximal end portion or lead connector end 10 of a conventional transvenous, bipolar pacing lead. The diameter of such a lead may be made a sufficiently small diameter to facilitate the lead's implantation into small veins such as those found in the coronary sinus region of the heart and to allow implantation of a plurality of leads into a single vessel for multi-site or multi-chamber pacing. It should be understood, however, that other lead designs may be used, for example, multi-polar leads have proximal ends portions that are bifurcated, trifurcated or have other branched configurations. While the lead with the proximal end shown in FIG. 1 is of the bipolar variety, there are unipolar leads that carry but a single electrode, and multi-polar leads that have more than two electrodes.

As is well known in the art, bipolar coaxial leads typically consists of a tubular housing of a biocompatible, biostable insulating material containing an inner multi-filar conductor coil that is surrounded by an inner insulating tube. The inner conductor coil is connected to a tip electrode on the distal end of the lead. The inner insulating tube is surrounded by a separate, outer multi-filar conductor coil that is also enclosed within the tubular housing. The outer conductor coil is connected to an anodal ring electrode along the distal end portion of the lead. The inner insulation is intended to electrically isolate the two conductor coils preventing any internal electrical short circuit, while the housing protects the entire lead from the intrusion of body fluids. These insulating materials are typically either silicone rubber or polyurethane. More recently, there have been introduced bipolar leads in which multi-filar cable conductors contained within multi-lumen housings are substituted for the conductor coils in order to reduce even further the overall diameter of the lead.

The proximal lead end portion or lead connector end 10 shown in FIG. 1 conforms to the IS-1 standard, comprising a pair of coaxial spaced-apart terminals including a tip terminal 12 and a ring terminal 14. The tip terminal 12 is electrically connected by means of the inner conductor coil to the tip electrode at the distal end of the lead, while the ring terminal 14 is electrically connected to the anodal ring electrode by means of the outer conductor coil. The tip and ring terminals of the lead may each be engaged by a conductive element or structure of the connector assembly (i.e., header) of the pulse generator as described below. The proximal end portion 10 further comprises spaced-apart pairs of seal rings 16 for preventing body fluids from reaching the electrical contacts. With the proximal end portion 10 of the lead inserted in a lead receptacle of a connector assembly (i.e., header) of a implantable pulse generator, the tip and ring terminals 12 and 14 are electrically coupled via the contacts and a feedthrough to the electronic circuits within the hermetically sealed can of the implantable pulse generator (e.g., cardiac pacemaker, implantable cardioverter defibrillator (ICD), or other implantable tissue stimulation and/or sensing device).

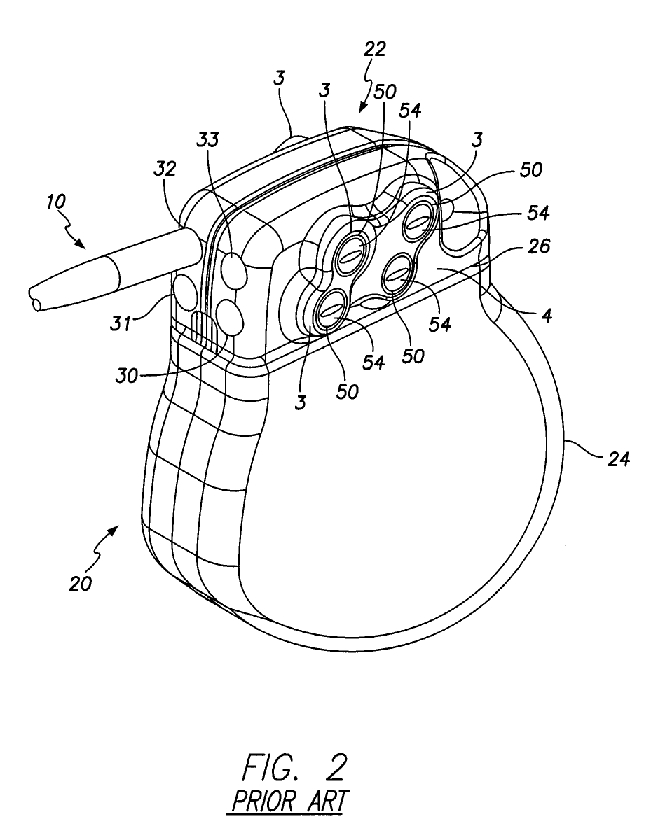

FIG. 2 shows a prior art multi-site or multi-chamber cardiac pacemaker/defibrillator unit (i.e., pulse generator) 20 incorporating a connector assembly 22. The cardiac pacemaker/defibrillator unit 20 is of a conventional design, including a hermetically sealed can or casing 24 enclosing the electronic components of the pacemaker/defibrillator unit with the connector assembly or header 22 mounted along a top edge 26 of the unit.

FIG. 2 illustrates that, in some embodiments, the connector assembly 22 may include four or more receptacles 30, 31, 32, and 33 for receiving the proximal ends of four leads. FIG. 2 also shows the proximal end portion 10 of a lead inserted in a receptacle 32. In other embodiments, the connector assembly 22 includes two receptacles comprising a single pair of receptacles (i.e., receptacles 30 and 31) for receiving the proximal ends of leads such as, for example, conventional bipolar leads and/or conventional cardioverting and/or defibrillating leads.

As can be understood from FIG. 2, the header 22 of the conventional pulse generator 20 has sidewalls that are not smooth and not generally planar on account of fisheye-style protrusions 3 that protrude outwardly from the rest of the sidewall surface 4 of the header 22. The fisheye-style protrusions 3 surround the setscrew bores 50 that are each shown in FIG. 2 as being filled or sealed by a septum 54. In this conventional pulse generator 20 of FIG. 2, the longitudinal axis of each setscrew that resides within a respective setscrew bore 50 is aligned with the longitudinal axis of the respective lead receptacle 30-33 such that the two longitudinal axes generally intersect in a perpendicular manner and could be said to reside in a single plane. Thus, the extreme inner tip end of the setscrew makes a normal or perpendicular, intersecting, tip point contact with the circumferential surface of a cylindrically shaped ring contact 14 or pin contact 12. To accommodate such an arrangement, the fisheye-type protrusions 3 are employed, and these fisheye-type protrusions can irritate the surround patient tissue.

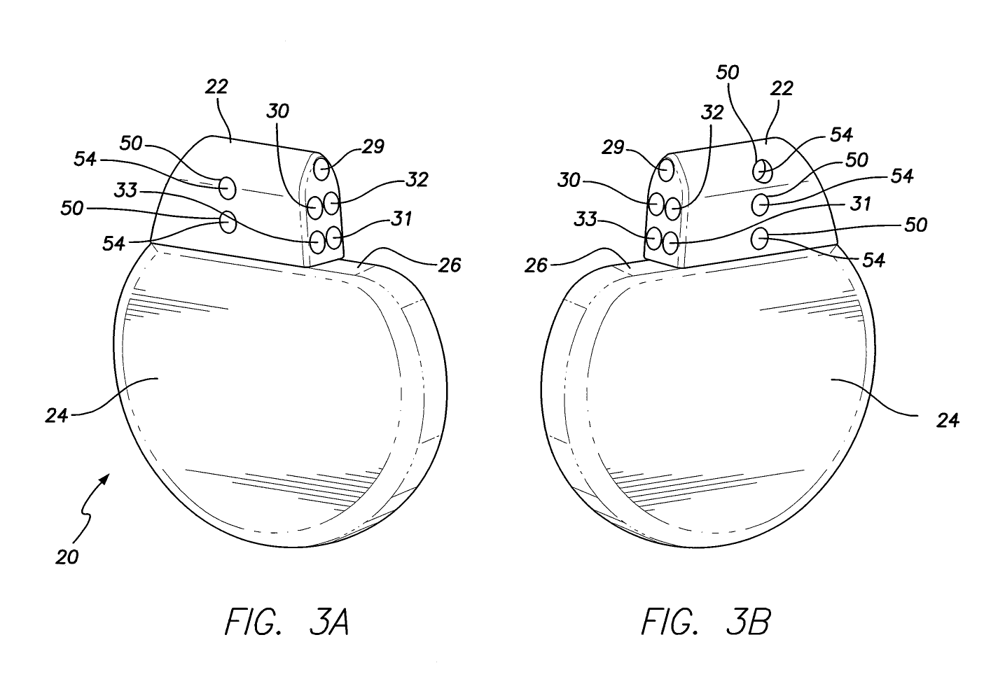

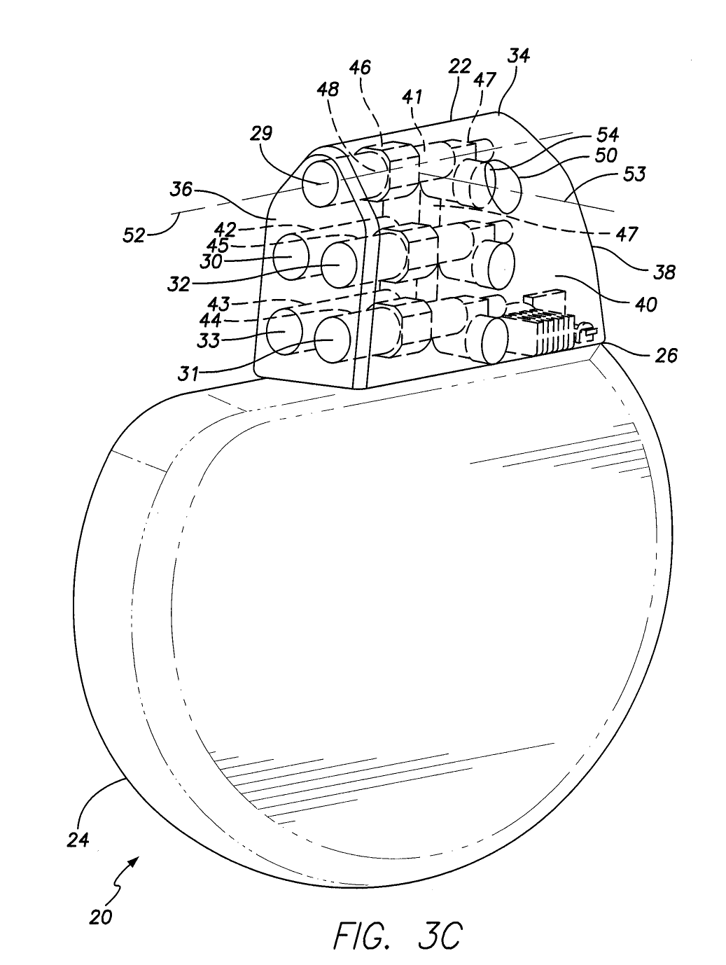

FIGS. 3A and 3B are isometric views of a new and novel pulse generator 20 as described herein, and FIG. 3C is an isometric view of the pulse generator 20 wherein internal components of the header 22 are shown in phantom lines. As shown in FIGS. 3A-3C, the connector assembly 22 includes five receptacles 29-33, however, depending on the embodiment, the number of receptacles may be greater or lesser than five. As illustrated in FIG. 3C, the components inside the exterior surface of the connector assembly 22 are shown in hidden lines. The connector assembly 22 includes a body 34 with a front or receptacle side 36, a rear side 38 and lateral sides 40 extending generally parallel to each other between the front side 36 and the rear side 38.

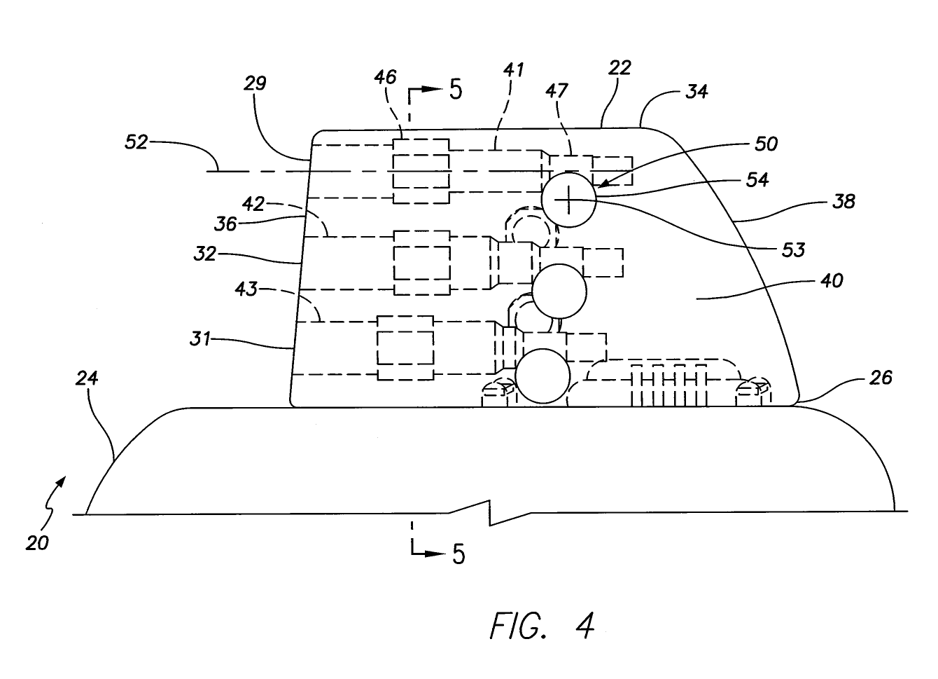

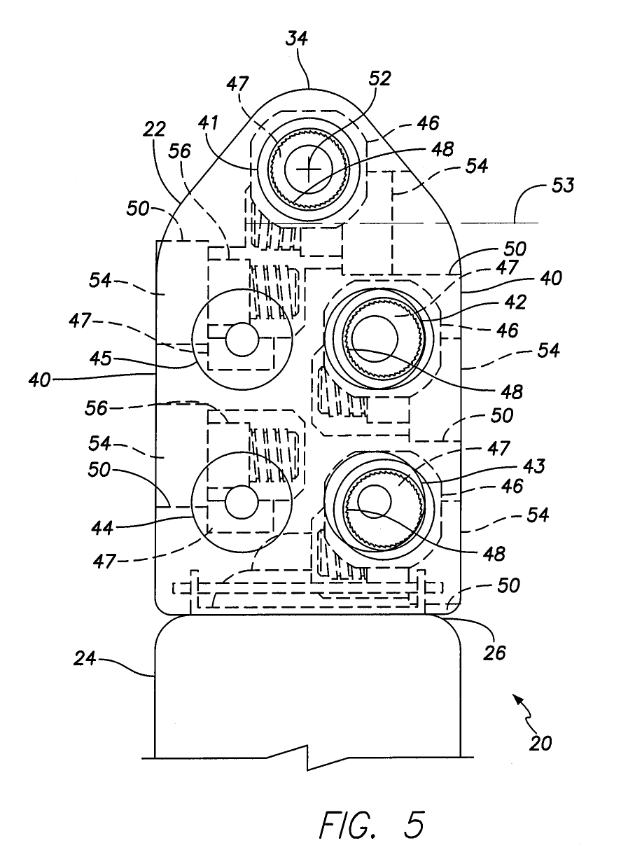

FIG. 4 is a side elevation view of the connector assembly 22 of FIGS. 3C, wherein the components inside the exterior surface of the connector assembly are shown in hidden lines. FIG. 5 is a transverse cross section elevation view of the connector assembly 22 as taken along section line 5-5 in FIG. 4, wherein the components inside the exterior surface of the connector assembly are shown in hidden lines. As can be understood from FIGS. 3C-5, the receptacles 29-33 are defined in the front side 36 and extend as generally parallel bores 41-45 through the body 34 of the connector assembly 22. As best understood from a comparison of the bores 41-43 depicted in FIG. 4 and the stepped cylindrical shape of the proximal lead end portion 10 illustrated in FIG. 1, at least some of the bores have a stepped cylindrical shape that is configured to matingly receive the proximal lead portion 10 depicted in FIG. 1. Other bores 44-45 may have similar or different configurations to matingly receive the proximal lead portion 10 depicted in FIG. 1 or other proximal lead portion configurations.

As shown in FIGS. 3C-5, at least some of the bores 41-43 include a conductive spring contact 46 and a conductive setscrew contact 47, and other bores 44-45 include only a conductive setscrew contact 47. As illustrated in FIGS. 3C and 5, the conductive spring contact 46 may employ a garter spring 48 that biases against the outer circumferential surface of the ring contact 14 of the lead connector end 10 of FIG. 1 to establish an electrical connection between the conductive spring contact 46 and the ring contact 14. The conductive set screw contact 47 serves to both secure a lead connector end 10 within the appropriate bore of the header 22 and to establish an electrical connection between the conductive set screw contact 47 and the pin contact 12 of the lead connector end 10 of FIG. 1.

As can be understood from a comparison of FIG. 4 and FIG. 1, when the proximal lead end portion 10 is received in one of the bores 41-43 as shown in FIG. 2, the tip terminal 12 is received by the most inward contact, which is a setscrew contact 47, and the ring terminal 14 is received by the most outward contact, which is a spring contact 46. The spring contacts 46 and setscrew contacts 47 electrically engage with the terminals 14 and 12 of the proximal lead end portion 10 to provide the generated electrical pulses from the can 24 of the pulse generator to the leads. In this manner, the electrical pulses are transmitted to the terminals of the proximal lead end 10 for transmission through the lead for delivery into the body via the electrode on the distal end of the lead.

As shown in FIGS. 3C and 5, setscrew bores 50 are defined in the lateral sides 40 and extend generally parallel into the body 34 of the connector assembly 22. As can be understood from FIGS. 3C-5, the longitudinal axis 52 of the top lead connector receiving bore 41 is generally perpendicular to the longitudinal axis 53 of the top setscrew bore 50, and these two perpendicular bores 41, 50 are offset such that the inner circumferential surfaces of the two bores 41, 50 generally tangentially intersect, as described in detail below. Similar relationships exist for each of the rest of the lead connector receiving bores 42-45 and the respective setscrew bores 50.

As indicated in FIGS. 3C-5, a septum 54 occupies the outer portion of each setscrew bore 50 so as to form a fluid tight seal barrier to isolate everything inward of the septum 54 from body fluids exterior to the pulse generator when implanted in a patient. A setscrew 56 occupies the inner portion of each setscrew bore 50 inside of the respective septum 54. Each setscrew 56 is threadably engaged with the respective conductive setscrew contact 47, as described in more detail below.

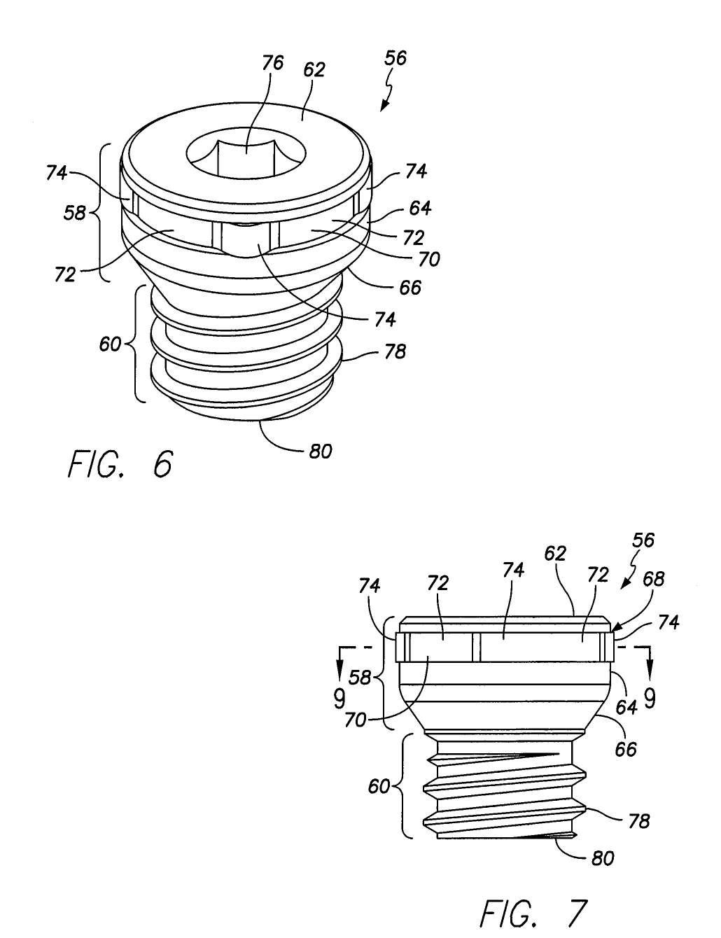

As illustrated in FIGS. 6, 7 and 8, which are, respectively, isometric, side elevation and top plan views of a typical setscrew 56 of the connector assembly 22, the setscrew 56 includes a head 58 and a threaded shaft 60 extending from the head 58. The head 58 includes a tool engagement face 62, a lateral cylindrical surface 64, and a tapered or beveled surface 66. The lateral cylindrical surface 64 forms a circumferential surface immediately adjacent the tool engagement face 62 that is generally perpendicular to the face 62. The tapered surface 66 forms a conical portion of the head 58 that transitions from the lateral cylindrical surface 64 to the threaded shaft 60.

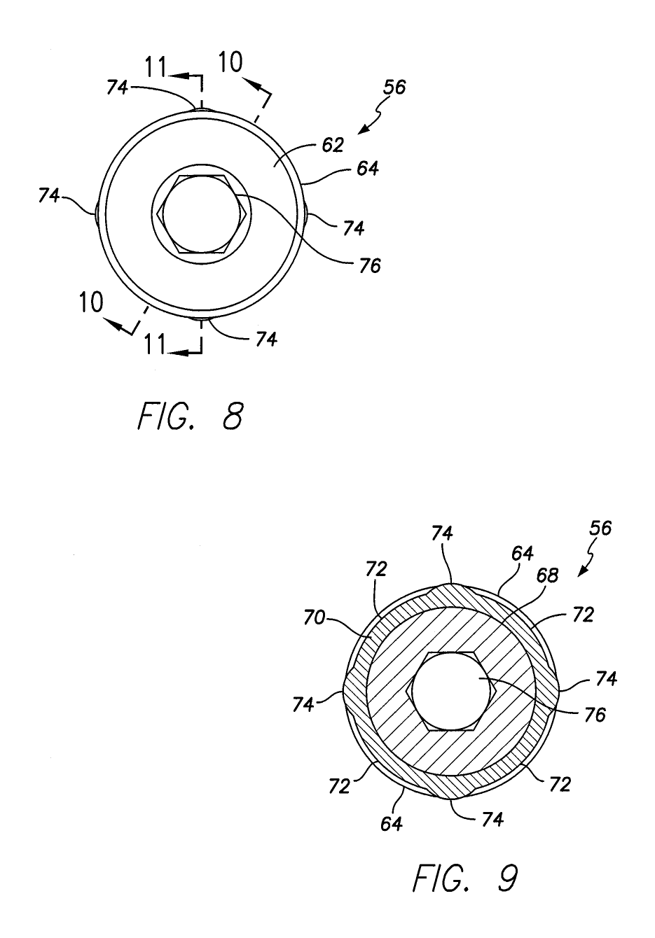

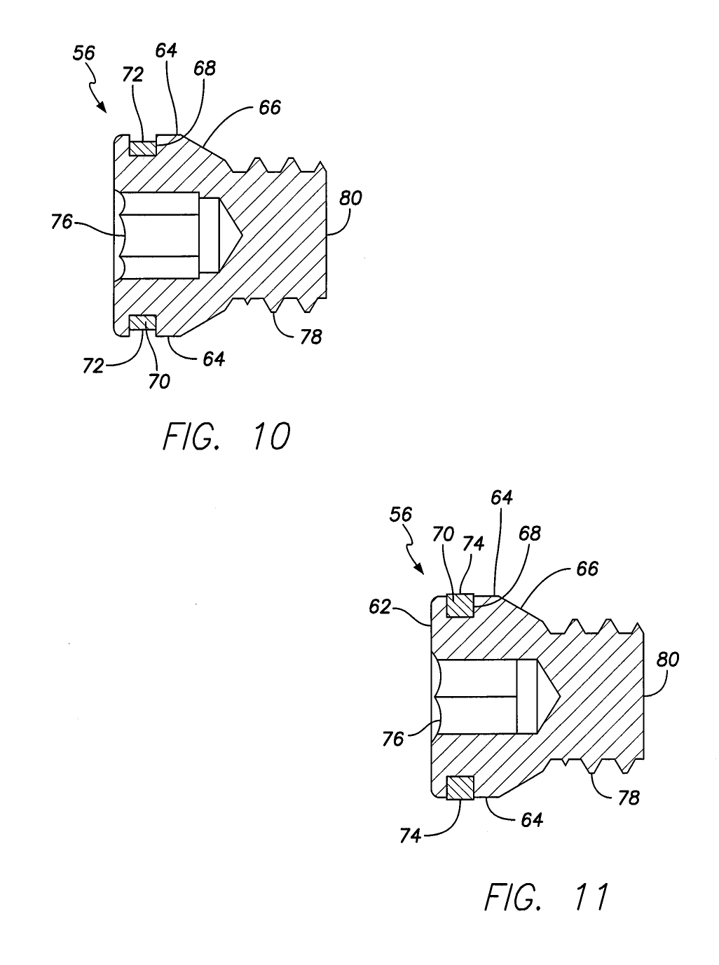

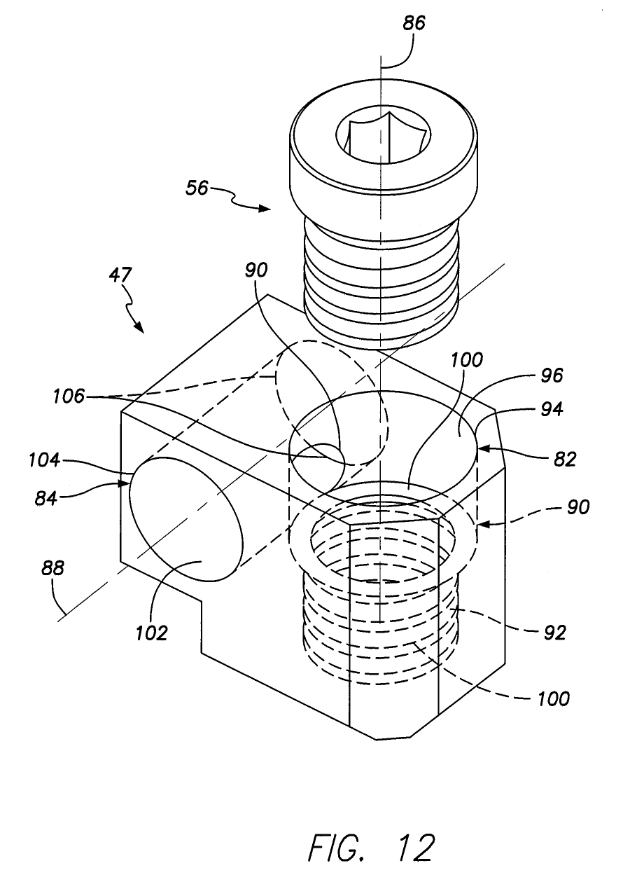

As shown in FIGS. 9, 10 and 11, which are, respectively, a transverse cross section through the head 58 as taken along section line 9-9 in FIG. 7, a longitudinal cross section through the setscrew as taken along section line 10-10 in FIG. 8, and another longitudinal cross section through the setscrew as taken along section line 11-11 in FIG. 8, a notch, groove or slot 68 is defined in the lateral cylindrical surface 64 so as to extend about the circumference of the head 58. An O-ring 70 occupies the groove 68 and includes an outer circumferential surface 72 with bumps or protrusions 74 that extend radially outward from the outer circumferential surface 72 of the O-ring 70. As illustrated in FIGS. 9 and 10, the outer circumferential surface 72 of the O-ring 70 is radially inward of the lateral cylindrical surface 64 of the head 58, and the protrusions 74 extend radially outward of the lateral cylindrical surface 64 of the head 58. Thus, when the setscrew 56 is fully received in the setscrew bore 50, the O-ring 70 does not form a fluid seal with the inner circumferential wall defining the setscrew bore 50 as the outer circumferential surface 72 of the O-ring 70 does not extend sufficiently radially outward from the lateral cylindrical surface 64 of the head 58 to contact the inner circumferential wall defining the setscrew bore 50. However, the protrusions 74 do extend sufficiently radially outward from the lateral cylindrical surface 64 of the head 58 to contact the inner circumferential wall defining the setscrew bore 50 when the setscrew 56 is fully received in the setscrew bore 50. As a result, the O-ring 70 and, more specifically, the protrusions 74 create an interference or abutting contact with the inner circumferential wall defining the setscrew bore 50 to act as an anti-rotation or rotation retarding feature for the setscrew 56 when received in the setscrew bore 50. Thus, protrusions 74 of the O-ring 70 abutting against the wall defining the setscrew bore 50 results in friction that acts to prevent the setscrew 56 from unintentionally rotating too far into or out of the setscrew bore 50.

As can be understood from FIGS. 8 and 9, the O-ring has four protrusions 74 that are evenly dispersed radially about the circumference of the head 58. For example, the protrusions can be located approximately 90 degrees apart from each other about the circumference of the head 58. In other embodiments, the protrusions 74 may be spaced apart a greater amount to have less protrusions (e.g., two protrusion at 180 degrees apart or three protrusions at 120 degrees apart) or space apart a lesser amount to have more protrusions (e.g., five protrusions at 72 degrees apart, six protrusions at 60 degrees apart, and so forth).

In one embodiment, each protrusion 74 extends radially outward from the lateral cylindrical surface 64 of the head 58 by a distance of between approximately 0.005″ and approximately 0.01″. In one embodiment, the O-ring 70 is formed of silicone, nylon, or Delrin. While such an O-ring 70 does not provide a fluid tight seal between the setscrew head and the inner circumferential surface of the setscrew hole 82, the protrusions provides resistance to the setscrew rotating within the setscrew hole, thereby acting to prevent the set screw 56 from unintentionally rotating too far into or out of the setscrew bore 50. The septum 54 provides the fluid seal that prevents body fluid from entering the setscrew bore and making contact with the electrified setscrew contact 47.

As shown in FIGS. 6 and 8-11, a tool engagement feature 76 is defined in the tool engagement face 62. The tool engagement feature 76 may be in the form of a hole, slot, nut or any other feature that can be engaged by an Allen wrench, screwdriver, wrench or other tool configured to cause a screw or bolt to rotate within a threaded opening, as is present in the conductive setscrew contact 47 as discussed below.

As depicted in FIGS. 6, 7, 10 and 11, the threaded shaft 60 includes threads 78 that helically extend from a point on the threaded shaft near the intersection of the conical portion 66 with the shaft 60 to an extreme free end 80 of the shaft 60. In one embodiment, the setscrew head and shaft are formed of Titanium, stainless steel, or MP35N.

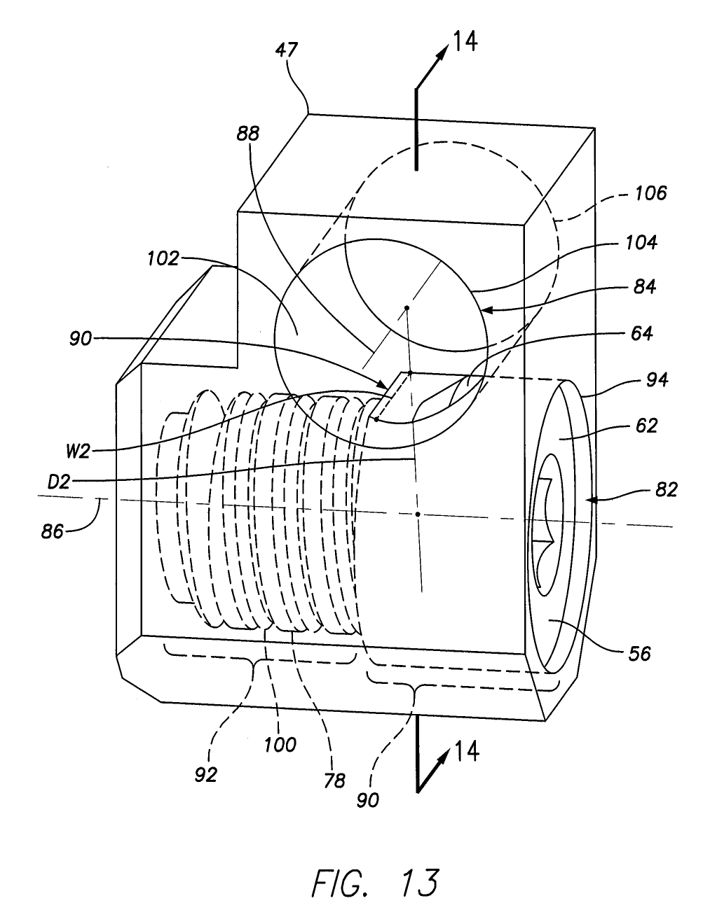

FIG. 12 is an isometric view of a typical conductive setscrew contact 47 of the connector assembly 22 with the setscrew 56 shown aligned with a threaded setscrew receiving opening 82 defined in the conductive setscrew contact 47. As shown in FIG. 12, the conductive setscrew contact 47 may have a one-piece body. The threaded setscrew receiving opening or setscrew hole 82 is defined in the one-piece body of the conductive setscrew contact 47. Also, a lead connector end portion receiving opening or lead connector hole 84 is defined in the one-piece body of the conductive setscrew contact 47. As can be understood from FIG. 12, the setscrew hole 82 and the lead connector hole 84 are oriented relative to each other such that their respective axes 86, 88 are generally perpendicular to each other and offset relative to each other such that portions of the two holes 82, 84 tangentially intersect to define a window 90 in the one-piece body 47 that opens into both holes 82, 84.

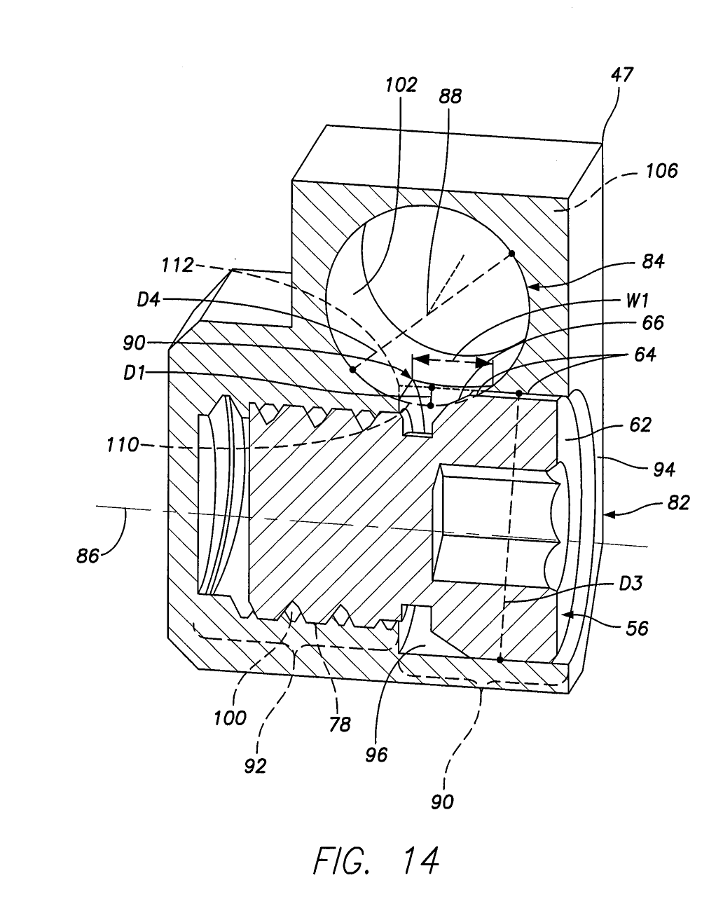

As illustrated in FIG. 12, the setscrew hole 82 includes a head portion 90 and threaded portion 92 that respectively correspond to the head 58 and threaded shaft 60 of the setscrew 56, as discussed above with respect to FIG. 6. The head portion 90 extends inward from an outer opening 94 of the setscrew hole 82 to transition into the threaded portion 92 substantially like a negative of the setscrew described above with respect to FIGS. 6-11. The head portion 90 includes a cylindrical circumferential inner surface 96, which defines a volume of the setscrew hole that receives the lateral cylindrical surface 64 and beveled surface 66 of the setscrew head when the setscrew is generally fully threadably received in the setscrew hole, as discussed below with respect to FIGS. 13 and 14. The threaded portion 92 includes threads 100 forming a female thread arrangement to threadably receive the male thread arrangement of the setscrew shaft.

As illustrated in FIG. 12, the lead connector hole 84 includes a cylindrical circumferential inner surface 102 that extends between a front outer opening 104 and a rear outer opening 106. The two holes 82, 84 can be said to generally tangentially intersect to define the window 90 in the one-piece body 47 that opens into both holes 82, 84. More specifically, and as will become more clear from FIGS. 13 and 14, the inner surface 96 of the setscrew hole 82 projects into the volume of the lead connector hole 84, and the inner surface 102 of the lead connector hole 84 projects into the volume of the setscrew hole 82. As a result, in the vicinity of where the inner surface 96 of the setscrew hole 82 intersects the inner surface 102 of the lead connector hole 84, the window 90 is created in the conductive setscrew contact 47 extending between the volumes of the two holes 82, 84.

FIG. 13 is an isometric view of the setscrew 56 threadably received in the conductive setscrew contact 47, and FIG. 14 is an isometric cross sectional elevation as taken along section line 14-14 of FIG. 13. As shown in FIGS. 13 and 14, were the inner surface 96 of the setscrew hole 82 to extend without interruption by the window 90, the inner surface 96 of the setscrew hole 82 would project into the volume of the lead connector hole 84. Similarly, where the inner surface 102 of the lead connector hole 84 to extend without interruption by the window 90, the inner surface 102 of the lead connector hole 84 would project into the volume of the setscrew hole 82. As a result and as indicated by the dashed line 110 indicating the hypothetical extension of the inner surface 102 of the lead connector hole 84 into the volume of the setscrew hole 82, the dashed line 110 being in the plane of section line 14-14 shown in FIG. 14 and along which the assembly depicted in FIG. 14 is sectioned, the volume of the lead connector hole 84 extends into the volume of the setscrew hole 82 via the window 90 defined by the intersection of the volumes of the two holes 82, 84. Similarly, as indicated by the dashed line 112 indicating the hypothetical extension of the inner surface 96 of the setscrew hole 82 into the volume of the lead connector hole 84, the dashed line 112 being in the plane of section line 14-14 shown in FIG. 14, the volume of the setscrew hole 82 extends into the volume of the lead connector hole 84 via the window 90 defined by the intersection of the volumes of the two holes 82, 84.

As indicated in FIGS. 13 and 14, when the setscrew 56 is sufficiently threadably received in the setscrew hole 82, the setscrew head conical portion 66 will enter the window 90 and project into the volume of the lead connector hole 84. Where a lead connector end 10 of FIG. 1 is fully received in the receptacle 32 as indicated in FIG. 2, the lead connector end tip or pin contact 12 will be aligned with the window 90. With the tip or pin contact 12 so positioned in the lead connector hole 84, the setscrew 56 can be increasingly threaded into the setscrew hole 82 to cause the conical surface 66 of the setscrew head 58 to increasingly impinge the lead connector end tip or pin contact 12 between the conical surface 66 and a region of the cylindrical inner circumferential surface 102 of the lead connector hole 82 generally opposite the window 90. With sufficient impingement of the tip or pin contact 12 via the conical surface 66, the lead connector end 10 will be mechanically secured within the receptacle 32 and an electrical connection between the tip or pin contact 12 and the conductive setscrew contact 47 will be mechanically created.

As indicated in FIG. 14, a width W1 of the window 90 transverse to the longitudinal axis 88 of the lead connector hole 84 is less than a diameter of the lead connector hole 84. Similarly, as shown in FIG. 13, a width W2 of the window 90 transverse to the longitudinal axis 86 of the setscrew hole 82 is less than a diameter of the setscrew hole 82. The width W1 may be between approximately 0.025″ and approximately 0.035″ for a lead connector hole 84 with a diameter at the window 90 of between approximately 0.045″ and approximately 0.07″. The width W2 may be between approximately 0.04″ and approximately 0.05″ for a setscrew hole 82 with a diameter at window 90 of between approximately 0.085″ and approximately 0.11″.

As can be understood from FIG. 13, the longitudinal axis 86 of the setscrew hole 82 is offset from the longitudinal axis 88 of the lead connector hole 84 by a distance D2 of between approximately 0.045″ and approximately 0.07″. Distance D2 can be calculated as the greatest distance perpendicular to both longitudinal axes 86, 88. As indicated in FIG. 14, the setscrew hole 82 in the region of the window 90 has a diameter D3 of between approximately 0.085″ and approximately 0.11″. The lead connector hole 84 in the region of the window 90 has a diameter D4 of between approximately 0.045″ and approximately 0.07″. In one embodiment, the distance D2 between the axes 86, 88 is approximately 0.064″ and the diameters D3, D4 of the holes 82, 84 are, respectively, 0.097″ and 0.054″.

As indicated in FIG. 14, a width W1 of the window 90 transverse to the longitudinal axis 88 of the lead connector hole 84 is less than a diameter of the lead connector hole 84. Similarly, as shown in FIG. 13, a width W2 of the window 90 transverse to the longitudinal axis 86 of the setscrew hole 82 is less than a diameter of the setscrew hole 82. The width W1 may be between approximately 0.025″ and approximately 0.035″ for a lead connector hole 84 with a diameter at the window 90 of between approximately 0.045″ and approximately 0.07″. The width W2 may be between approximately 0.04″ and approximately 0.05″ for a setscrew hole 82 with a diameter at window 90 of between approximately 0.085″ and approximately 0.11″.

In one embodiment, the setscrew connector body 47 is a unitary-piece construction wherein the holes 82, 84 are milled, drilled or otherwise machined in the unitary-piece body. The body 47 may be formed of an electrically conductive metal such as titanium, stainless steel, MP35N, or palladium. In other embodiments, the body 47 is formed via other methods, such as, for example, molding/forming, etc.

As can be understood from FIGS. 3C, 5, 13 and 14, the setscrew 56 and setscrew contact 47 may be configured such that the setscrew is fully recessed within the setscrew hole 82 in the setscrew contact 47 when the setscrew is in a position within the setscrew hole that will allow the setscrew to secure a lead connector end within the lead connector hole 84. As a result and as shown in FIG. 5, the header 22 has a smooth low profile with a more narrow appearance. The header 22 may have a transverse width that is generally the same as the transverse width of the can 24. Also, the header 22 may have a generally smooth lateral sidewall 40 with little, if any, topographical discontinuity that protrudes outwardly from the smooth lateral sidewall 40. Such a header configuration is advantageous in that there is greater patient comfort and reduced complications (e.g., pocket erosion) associated with such a header and associated can implanted in a patient. A header with a smooth sidewall is also preferred by physicians since it increases lead tip visibility during implant procedures, as can be understood from FIG. 4.

The foregoing merely illustrates the principles of the invention. Various modifications and alterations to the described embodiments will be apparent to those skilled in the art in view of the teachings herein. It will thus be appreciated that those skilled in the art will be able to devise numerous systems, arrangements and methods which, although not explicitly shown or described herein, embody the principles of the invention and are thus within the spirit and scope of the present invention. From the above description and drawings, it will be understood by those of ordinary skill in the art that the particular embodiments shown and described are for purposes of illustrations only and are not intended to limit the scope of the present invention. References to details of particular embodiments are not intended to limit the scope of the invention.

What is claimed is:

1. An implantable medical pulse generator for administering electrotherapy via an implantable medical lead having a lead connector end on a proximal end of the lead, the lead connector having a terminal, the pulse generator comprising:
a can; and
a header coupled to the can, the header comprising:
a lead connector end receiving receptacle; and
a conductive setscrew contact associated with the lead connector end receiving receptacle and configured to be in electrical contact with the terminal of the first lead connector end when the lead connector end is inserted into the lead connector end receiving receptacle,
wherein the conductive setscrew contact comprises:
a lead connector hole defined at least in part by a circumferential inner surface and including a longitudinal axis, the lead connector hole being generally aligned with the lead connector end receiving receptacle;
a setscrew hole defined at least in part by a circumferential inner surface and including a longitudinal axis generally perpendicular to and offset away from the longitudinal axis of the lead connector hole, the circumferential inner surface of the setscrew hole being nearly tangential with the circumferential inner surface of the lead connector hole such that a window is defined in the circumferential inner surface of the setscrew hole and the circumferential inner surface of the lead connector hole by the overlapping of a volume of the lead connector hole with a volume of the setscrew hole; and
a setscrew comprising a threaded shaft extending from a head, wherein a portion of the head resides within the window to protrude from the setscrew hole into the lead connector hole when the setscrew is threadably received in the setscrew hole.

2. The pulse generator of claim 1, wherein a width of the window transverse to the longitudinal axis of the lead connector hole is less than a diameter of the lead connector hole.

3. The pulse generator of claim 1, wherein a width of the window transverse to the longitudinal axis of the setscrew hole is less than a diameter of the setscrew hole.

4. The pulse generator of claim 1, wherein the setscrew includes a conical portion which is the portion of the head residing with the window to protrude from the setscrew hole into the lead connector hole when the setscrew is threadably received in the setscrew hole.

5. The pulse generator of claim 1, wherein the setscrew includes a cylindrical portion which is the portion of the head residing with the window to protrude from the setscrew hole into the lead connector hole when the setscrew is threadably received in the setscrew hole.

6. The pulse generator of claim 5, wherein the setscrew further includes a groove defined in an outer circumferential surface of the cylindrical portion of the head of the setscrew, the groove being occupied by an O-ring with an outer circumferential boundary that does not radially extend past the outer circumferential surface of the cylindrical portion except at radially spaced apart distinct protrusions of the O-ring that do radially extend past the outer circumferential surface of the cylindrical portion.

7. The pulse generator of claim 6, wherein the only part of the O-ring that contacts the inner circumferential surface of the setscrew hole when the setscrew is threadably received in the setscrew hole are the protrusions of the O-ring.

8. The pulse generator of claim 7, wherein the contact of the O-ring protrusions with the inner circumferential surface of the setscrew hole does not create a fluid tight seal about the setscrew head.

9. The pulse generator of claim 8, wherein the contact of the O-ring protrusions with the inner circumferential surface of the setscrew hole creates an arrangement that helps to resist the rotation of the setscrew within the setscrew hole.

10. The pulse generator of claim 1, wherein the header further includes a setscrew bore generally aligned with the setscrew hole.

11. The pulse generator of claim 10, further comprising a septum configured to create a fluid tight seal with an inner circumferential surface of the setscrew bore, the septum being located in the setscrew bore outward from the setscrew head.

12. The pulse generator of claim 1, wherein the volume of the setscrew hole overlaps with the volume of the of the lead connector hole by a distance of between approximately 0.025″ and approximately 0.05″.

13. The pulse generator of claim 1, wherein the longitudinal axis of the setscrew hole is offset from the longitudinal axis of the lead connector hole by a distance of between approximately 0.045″ and approximately 0.07″.

14. The pulse generator of claim 13, wherein the setscrew hole in the region of the window has a diameter of between approximately 0.085″ and approximately 0.11″, and the lead connector hole in the region of the window has a diameter of between approximately 0.045″ and approximately 0.07″.

15. The pulse generator of claim 1, wherein the longitudinal axis of the setscrew hole is offset from the longitudinal axis of the lead connector hole by a distance of approximately 0.064″, the setscrew hole in the region of the window has a diameter of approximately 0.097″, and the lead connector hole in the region of the window has a diameter of approximately 0.054″.

16. An implantable medical pulse generator for administering electrotherapy via an implantable medical lead having a lead connector end on a proximal end of the lead, the lead connector having a terminal, the pulse generator comprising:
a can; and
a header coupled to the can, the header comprising:
a lead connector end receiving receptacle; and
a conductive setscrew contact associated with the lead connector end receiving receptacle and configured to be in electrical contact with the terminal of the first lead connector end when the lead connector end is inserted into the lead connector end receiving receptacle,
wherein the conductive setscrew contact comprises:
a lead connector hole defined at least in part by a circumferential inner surface and including a longitudinal axis, the lead connector hole being generally aligned with the lead connector end receiving receptacle;
a setscrew hole defined at least in part by a circumferential inner surface and including a longitudinal axis generally perpendicular to and offset away from the longitudinal axis of the lead connector hole; and
a setscrew comprising a threaded shaft extending from a head, wherein a portion of the head protrudes from the setscrew hole into the lead connector hole when the setscrew is threadably received in the setscrew hole,
wherein the setscrew further includes a groove defined in an outer circumferential surface of a cylindrical portion of the head of the setscrew, the groove being occupied by an O-ring with an outer circumferential boundary that does not radially extend past the outer circumferential surface of the cylindrical portion except at radially spaced apart distinct protrusions of the O-ring that do radially extend past the outer circumferential surface of the cylindrical portion.

17. The pulse generator of claim 16, wherein the only part of the O-ring that contacts the inner circumferential surface of the setscrew hole when the setscrew is threadably received in the setscrew hole are the protrusions of the O-ring.

18. The pulse generator of claim 17, wherein the contact of the O-ring protrusions with the inner circumferential surface of the setscrew hole does not create a fluid tight seal about the setscrew head.

19. The pulse generator of claim 18, wherein the contact of the O-ring protrusions with the inner circumferential surface of the setscrew hole creates an arrangement that helps to resist the rotation of the setscrew within the setscrew hole.

20. The pulse generator of claim 16, wherein the header further includes a setscrew bore generally aligned with the setscrew hole.

21. The pulse generator of claim 20, further comprising a septum configured to create a fluid tight seal with an inner circumferential surface of the setscrew bore, the septum being located in the setscrew bore outward from the setscrew head.
